(19) United States
(12) Patent Application Publication
(10) Pub. No.: US 2007/0098790 A1
(43) Pub. Date: May 3, 2007

(54) Nutritional supplement for the enhancement of the health of the liver

(76) Inventor: David Jiang, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/263,585

(22) Filed: Oct. 31, 2005

(51) Int. Cl. A61K 38/05; A61K 31/375; A61K 31/198; A61K 31/355; A61K 9/22

(52) U.S. Cl. 424/468; 514/18; 514/458; 514/474; 514/562

(57) ABSTRACT

Compositions, systems, and methods of enhancing the health of the liver are disclosed. The synergistic effect of N-acetylcysteine, glutathione, vitamin C and vitamin E is to counteract the toxic effects of analgesics such as acetaminophen on the liver. Also disclosed is an enteric coating material which may be used with the disclosed nutritional supplement and/or other supplements, nutraceuticals, and pharmaceuticals.

### BACKGROUND OF THE INVENTION

1. Field of the Invention

This invention relates to compositions and methods for improving the health of the liver such as by counteracting the toxic effects of analgesics such as acetaminophen. This invention also relates to a tablet containing compositions for improving the health of the liver which is includes a coating that allows for efficient absorption of the composition into the body of a mammal.

2. Description of the Related Art

Drug toxicity is a major problem in the healthcare industry, especially in pre-adolescent and alcoholic patients. Acetaminophen, for example, has been shown to cause hepatotoxicity in large doses. Since acetaminophen is called by many names and is contained in over 100 products, inadvertent overdoses commonly occur. Often, young children taking prescription drugs that contain acetaminophen are unknowingly given additional acetaminophen by their parents in the form of other over the counter products. Acute overdosage of acetaminophen can result in potentially fatal hepatic necrosis.

Glutathione is a tripeptide found in all animal cells at relatively high concentrations. Its depletion is implicated in the mechanism of many diseases and damage to organs such as the liver. Acetaminophen has in fact been shown to decrease glutathione levels in cells. As a method of treatment, many attempts have been made to increase glutathione levels in the body with limited success.

### SUMMARY OF THE INVENTION

In accordance with one embodiment, there is provided a supplement composition. The supplement may be used for treating or preventing the toxic effects of analgesics on the liver of a mammal. The supplement composition comprises N-acetylcysteine, glutathione, vitamin C, vitamin E and a pharmaceutically acceptable carrier, wherein the molar ratio of N-acetylcysteine to glutathione is preferably between 1:1 and 20:1.

In accordance with another embodiment, there is provided a supplement composition consisting essentially of N-acetylcysteine, glutathione, vitamin C, and vitamin E, wherein the molar ratio of N-acetylcysteine to glutathione is preferably between 1:1 and 20:1.

In accordance with another embodiment, there is provided a supplement composition comprising N-acetylcysteine, glutathione, vitamin C, vitamin E and a pharmaceutically acceptable carrier, wherein the molar ratio of N-acetylcysteine to glutathione is preferably between 1:1 and 20:1 and wherein at least a portion of the composition is coated by an enteric coating material.

Preferred embodiments may include one or more of the following: at least a portion of the N-acetylcysteine and glutathione are coated with an enteric coating material; the enteric coating material comprises a film-forming polymer and a plasticizer; the film-forming polymer comprises sodium alginate; and/or the plasticizer comprises a polyalkylene glycol, polyalkylene glycol copolymer, polyalkylene oxide, and/or polyalkylene oxide copolymer, preferably polyethylene glycol.

In accordance with one embodiment, there is provided a controlled-release supplement composition comprising N-acetylcysteine, glutathione, vitamin C, vitamin E, and a pharmaceutically acceptable carrier, wherein the molar ratio of N-acetylcysteine to glutathione is preferably between 1:1 and 20:1 and at least a portion of the N-acetylcysteine and glutathione are coated with one or more compounds adapted to cause delivery of the N-acetylcysteine and glutathione in the small intestine. In a preferred embodiment, the one or more compounds comprise a film-forming polymer and a plasticizer.

In accordance with another embodiment, there is provided a supplement comprising a core component comprising N-acetylcysteine and glutathione, coated by an enteric coating composition comprising a film-forming polymer and a plasticizer; and a second component comprising vitamin C and vitamin E, wherein the second component substantially surrounds the core component and wherein the molar ratio of N-acetylcysteine to glutathione in the composition is preferably between 1:1 and 20:1. In related embodiments, the supplement is further characterized by one or more of the following: the core component further comprises vitamin C and/or vitamin E, at least about 50% of the total vitamin C and vitamin E in the composition is present in the core component; at least about 50% of the NAC and glutathione in the composition is present in the core component; the second component further comprises N-acetylcysteine and/or glutathione; the film-forming polymer comprises sodium alginate; and/or the plasticizer comprises a polyalkylene glycol.

In accordance with another embodiment, there is provided an enteric coating composition comprising a powder mixed with water or aqueous solution, wherein the powder comprises sodium alginate and a water-soluble plasticizer. In another embodiment, there is provided an enteric coating composition, comprising water and a powder comprising sodium alginate and a water-soluble plasticizer, wherein the composition comprises about 2.5 to about 50.0 grams powder per liter of water. In related embodiments, the coating material is further characterized by one or more of the following: the plasticizer comprises a polyalkylene glycol, polyalkylene glycol copolymer, polyalkylene oxide, and/or polyalkylene oxide copolymer, the plasticizer comprises polyethylene glycol, the sodium alginate comprises about 50-95% by weight of the dry powder; and/or the coating further comprises one or more pigments or colorants.

### DETAILED DESCRIPTION OF THE PREFERRED EMBODIMENTS

It is noted that, as used in this specification and the appended claims, the singular forms “a,” “an,” and “the” include plural referents unless the context clearly dictates otherwise.

In describing and claiming the preferred embodiments, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the preferred embodiments.

Embodiments of the invention relate to enhancing the health of the liver of a mammal, including counteracting the toxic effect of analgesics such as acetaminophen, by the novel synergistic administration of N-acetylcysteine (NAC), glutathione, vitamin C and vitamin E. The combination of these components provides the surprisingly advantageous effect of preventing and treating toxicity of the liver caused by analgesics more efficiently and completely than prior compositions.

In some embodiments of the invention, the NAC, glutathione, vitamin C and vitamin E are present in a composition that can be in tablet (including caplets), capsule, liquid, intravenous or other forms. One preferred form is a tablet for oral administration. Methods and apparatus for pressing a mixture of dry ingredients or a mixture of dry and liquid ingredients into tablets are well known in the art.

NAC and glutathione depletion have been implicated in many mechanisms of disease, including liver toxicity caused by overdoses of analgesics. In one embodiment, the NAC and glutathione are present in the composition at a molar ratio of between about 1:1 to about 20:1, preferably between about 3:1 and about 7:1 (NAC:glutathione). Since NAC is a precursor to glutathione in the body, providing an excess of NAC allows it to be available as a reservoir for the body to generate more glutathione in the future if it is depleted. In this way, the inventive composition is effective at both treating and preventing the toxic effects of analgesics on the liver of a mammal. In one embodiment, the glutathione is provided in excess of NAC at a ratio of between about 1:1 and about 1:3 (NAC;glutathione).

In some embodiments, the composition comprises between about 1 mg to about 10,000 mg of NAC, including between about 400 mg to about 8000 mg of NAC and between about 100 mg and about 800 mg of NAC. In some embodiments, the composition comprises between about 1 mg to about 5000 mg of glutathione, including between about 30 mg to about 1000 mg of glutathione and between about 50 mg to about 200 mg of glutathione.

Preferred compositions comprise vitamin E and vitamin C in addition to the NAC and glutathione. Vitamin E and vitamin C are present in amounts effective in preventing oxidative damage to the NAC, glutathione, and/or liver cells. In a preferred embodiment, the vitamin E is present at an amount of 30 IU. In one embodiment, a composition comprises about 1 IU to about 600 IU of vitamin E, including about 1 IU to about 120 IU and about 5 IU to about 60 IU. In a preferred embodiment, the vitamin C is present at an amount of 30 mg. In one embodiment, a composition comprises about 1 mg to about 700 mg of vitamin C, including about 1 mg to about 100 mg and about 5 mg to about 60 mg.

In some embodiments, the compositions include other active ingredients other than NAC, glutathione, vitamin E and vitamin C. In other embodiments, the compositions consist essentially of NAC, glutathione, vitamin E and vitamin C: that is, these are the only four active ingredients and the remainder of the composition includes one or more pharmaceutically acceptable carriers such as excipients, diluents, enteric coatings, delivery agents, fillers, and the like.

One or more pharmaceutically acceptable carriers may be used in any of the present compositions and formulations. As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed in order to prepare a particular composition. The use of such media and agents for substances is well known in the art. Except insofar as any conventional media or agent is incompatible with the active ingredients, its use in preferred embodiments is contemplated. Other ingredients known to affect the manufacture of preferred embodiments can include flavorings, sugars, proteins and/or modified starches, as well as fats and oils.

The compositions disclosed herein, and/or portions thereof, may be formulated to create sustained release formulations. Sustained release formulations, and methods of making such formulations, are well known in the art. A sustained release formulation regulates the release of the components and allows for a steady absorption by the body over time providing a more level concentration of one or more actives to be maintained in the body over a greater period of time. This is advantageous because it allows for a larger time frame for the anti-oxidant and/or other beneficial effects on the liver which means a longer protection from and treatment of damage such as that resulting from analgesic-induced toxicity.

Preferred embodiments intended for oral administration may result in delivery of a much lower actual dose of the NAC and/or glutathione because the compounds, especially glutathione, are unstable in the acidic conditions of the stomach and may be destroyed prior to absorption. Therefore, in some embodiments for oral administration, the composition, or a portion thereof, is in the form of a delayed or controlled release formulation which allows targeting of release of the components at a particular time and location within the body. This is advantageous because it would allow for the protection of some or all of the NAC and/or glutathione from destabilization in the stomach. In this way, the portions of the NAC and glutathione which are formulated for delayed release can be transported generally intact to the small intestine where they are absorbed.

Enteric coating provides one possible method for bringing NAC and/or glutathione to the small intestine. Use of an enteric coating to protect some or all of the NAC and/or glutathione in a composition can provide controlled or delayed release of the coated materials, meaning that the release of the enterically coated materials does not occur immediately, but instead occurs at a later time and further down the digestive tract. Use of enteric coatings could also provide sustained release of components, in that use of multiple layers of actives, at least some of which are enterically coated, can allow for one or more actives to be released in two or more different stages following administration. When enteric coatings are used, the time of release may be adjusted and varied by changing the type, number and/or thickness of the enteric coating layer.

In some embodiments, the entire inventive composition is present inside the enteric coating. In others, at least part of the composition is present in one or more outer layers in an immediate release form which partially or fully surrounds or is otherwise physically associated with a core comprising the remainder of the composition which is surrounded by an enteric coating. In some embodiments, some portion or all of the vitamin C and vitamin E are present outside of the enteric coating to provide further anti-oxidant effects to the body.

However, NAC and glutathione may have some instability in oxidative environments such as the small intestine, albeit substantially less than their instability in the stomach. Vitamin C and vitamin E are more stable than NAC and glutathione and can protect these compounds from oxidative damage in the small intestine. Accordingly, in some embodiments, vitamin C, vitamin E, and/or one or more other antioxidants are included in at least an enterically coated portion of the composition along with NAC and/or glutathione. In a preferred embodiment, vitamin C and vitamin E are present in amounts effective to protect the NAC and glutathione from oxidative damage in the small intestine thereby enhancing absorption. In one embodiment, at least about 50%, including at least about 75% and at least about 90% of the total vitamin C and/or the total vitamin E of the composition is contained within an enterically coated core, along with at least about 50%, including at least about 75% and at least about 90% of the total NAC and/or the total glutathione of the composition. Inclusion of one or more antioxidants in an enteric coated layer or core including NAC and/or glutathione can allow for more effective absorption in the small intestine.

For example, a composition in tablet form may include multiple layers, such as the following, where the layers are listed from the exterior to the interior of the tablet: an first layer or exterior coating of a gelatin to assist in swallowing of the composition, but which dissolves rapidly in the stomach; a second layer for substantially immediate release in the stomach comprising the vitamin E, vitamin C and about 10-20% of the glutathione and NAC; third layer of an enteric coating; a fourth layer comprising about 60-70% of the glutathione and about 30-40% of the NAC; a fifth layer comprising an enteric coating; and a sixth, innermost layer comprising about 10-30% of the glutathione and about 40-70% of the NAC.

Suitable enteric coatings include any which provide for passage of a substantial amount of the coated portion of the composition through the stomach to the intestine without being exposed or damaged by the environment of the stomach. A great many formulations of enteric coatings are known in the art, and may be used in accordance with embodiments which include enteric coatings.

Application of coating materials, including enteric coating materials, to a tablet can be done by any suitable method, including those presently known in the art. One coating method is called a film coating process. In a film coating process an aliquot of solution or suspension of coating material is sprayed onto a batch of tablets which are gently tumbled in a coating pan or vessel to obtain a more even distribution of coating material on the tablets. Warm or hot air is flowed over the tablets following application or generally constantly to aid in drying of the coating. The application of coating material is repeated as many times as necessary to achieve a complete coating or the desired thickness of coating.

Preferred Enteric Coating Material

In some embodiments, a preferred enteric coating as disclosed herein may be used with the compositions to enhance liver health, as well as with other compositions where enteric coating is desired. These preferred enteric film-coating compositions overcome one or more of the problems of the prior art enteric coating by providing a natural derived edible enteric film-coating dry powder that is substantially complete, and needs only to be dissolved in an aqueous solution to be ready for coating tablets. The film coating composition can be shipped in dry powder form, which is more efficient than shipping a coating that includes solvent. The film-coating enteric composition can also be stored in dry form which avoids problems of evaporation, attack by bacteria, and the deleterious effects of heat and/or cold on a liquid dispersion.

In some embodiments, the enteric film coating is naturally derived and edible and comprises a film-forming polymer and a plasticizer. A coating solution or suspension can be made by mixing this dry powder with water or aqueous solution. The water or aqueous solution is preferably substantially free of volatile organic solvents or compounds. In a preferred embodiment, the coating solution or suspension is made by combining powder and water, preferably about 2.5 to 50.0 grams of powder per liter of water, including 5 to 20 grams per liter and then mixing the powder and water until a solution or generally uniform suspension is formed.

The film-forming polymer can be, for example, sodium alginate with a preferred viscosity less than about 1500 for a 1.25% solution by weight in water, and a preferred particle size above about 50 microns including above about 100 microns, above about 150 microns, above about 200 microns and above about 250 microns. The film-forming polymer comprises preferably about 50-95% by weight of the dry powder, including about 75-85% by weight of the dry powder.

In one embodiment, the dry powder of the coating further comprises a water soluble plasticizer. When the dry powder is mixed into water, the water soluble plasticizer dissolves to form solvated molecules of the plasticizer which can react more efficiently with the film-forming polymer dissolved in the coating solution or suspension to produce a more effective enteric coating when applied to tablets. The water soluble plasticizer comprises preferably about 1-50% by weight of the dry powder, including about 2-15% and about 5-25% by weight of the dry powder. Preferred plasticizers include polyalkylene glycols (e.g. polyethylene glycol, polypropylene glycol, copolymers of polyalkylene glycols), polyalkylene oxides (e.g. polyethylene oxide, polypropylene oxide, copolymers of polyalkylene oxides), and glycerol, sorbitol, propylene glycol, diethyl phthalate, triacetin.

The enteric film coating may optionally include one or more of pigment particles, coloring agents, and anti-caking agents. The total amount of optional ingredients, if present, preferably comprises about 0.1-15% by weight of the dry powder, including about 0.1-1%, about 5-15%, and about 1-10% by weight of the dry powder. The amount of each individual optional ingredient, if present, preferably comprise about 0.1-15% by weight of the dry powder, including about 0.1-1%, about 5-15%, and about 1-10% by weight of the dry powder. In some embodiments, pigment particles, if present, may be any pigment commonly used in making coating dispersions for nutraceutical, pharmaceutical and other tablets. For example, the pigments may be FD&C and D&C Lakes, titanium dioxide, magnesium carbonate, talc, pyrogenic silica, iron oxides, channel black, and insoluble dyes. Also, the pigments can be natural pigments such as riboflavin, carmine 40, curcumin, and annatto. Combinations of two or more such pigments may also be used.

In one embodiment, the film-forming polymer comprises sodium alginate which makes the coating solution or suspension thicker and thereby inhibits settlement of any solids which may be present in the coating solution or suspension and acts as a suspending agent and a film-former. Sodium alginate, although it is water soluble, is not soluble at low pH such as that found in gastric juices, and thus the sodium alginate does not interfere with enteric performance by disintegrating in the gastric juices of the stomach. The sodium alginate can also act as a film-former during the spraying operation to assist in providing an even coating to oral tablets.

In one embodiment of the invention, the method of making and applying the inventive enteric coating to tablets is as follows. First, the ingredients of the coating dry powder are mixed together. Then the coating dry powder is dispersed into water to form a coating solution (generally without pigments) or suspension (usually with pigments for color coating). The coating dry powder is preferably added slowly to the water with constant mixing. In one embodiment, the addition and mixing occur for about 60 to 90 minutes. Following addition and mixing, the coating solution or suspension is ready for use.

In some embodiments, certain tablet configurations or core compositions may be coated with a sub-coat prior to the application of the inventive enteric solution or suspension. Sub-coating, such as with a solution or suspension of a water soluble polymer (e.g. polyalkyleneglycol, starch, or gelatin, hydroxyl methylcellulose, hydroxypropyl cellulose) can provide a smooth surface on an otherwise rough core. It can also aid in reducing or preventing edge chipping and tablet abrasion which can cause inaccurate dosing or rejection of product for cosmetic reasons.

The disclosure below is of specific examples setting forth preferred compositions, systems, and methods. These examples are not intended to limit the scope, but rather to exemplify preferred embodiments. The following examples relate to preferred embodiments.

### EXAMPLE 1

A dry mix of:

| | | Percentage |
|---|---|---|
| Ingredients | grams | of dry powder |
| Sodium Alginate | 82.5 | 82.5% |
| Polyethylene glycol 3350 | 7.52 | 7.52% |
| FD&C Yellow NO. 6 Aluminum Lake | 5.05 | 5.05% |
| D&C Yellow No. 10 Aluminum Lake | 4.93 | 4.93% |

is prepared by first blending the ingredients in a twin shell blender until uniform and then passing them through a Fitzmill to disperse any small agglomerates.

Using a high speed agitator, 1.5 grams of the dry mix are suspended in 100 milliliters of de-ionized water. After stirring for 60 minutes, the coating suspension is ready for application to tablets.

### EXAMPLE 2

A dry mix is made in accordance with Example 1 except that 6.52 grams (6.6%) of polyethylene glycol 3350 are used instead of 7.52 grams.

### EXAMPLE 3

A dry mix is made in accordance with Example 1 except that 10 grams of polyethylene glycol 3350 (9.8%) are used instead of 7.52 grams.

### EXAMPLE 4

A dry mix is made in accordance with Example 1 except that 7.52 grams (7.52%) of polyethylene glycol 8000 are substituted for the polyethylene glycol 3350.

### EXAMPLE 5

A dry mix is made in accordance with Example 1 except that 10 grams (9.8%) of polyethylene glycol 8000 are substituted for the polyethylene glycol 3350.

### EXAMPLE 6

A dry mix is made in accordance with Example 1 except that 75.5 grams (81.2%) of sodium alginate are used instead of 82.50 grams.

### EXAMPLE 7

A dry mix is made in accordance with Example 1 except that 80 grams (82.1%) of sodium alginate are used instead of 82.5 grams.

### EXAMPLE 8

A dry mix is made in accordance with Example 1 except that 85 grams (82.9%) of sodium alginate are used instead of 82.5 grams.

### EXAMPLE 9

A dry mix is made in accordance with Example 1 except that 20 grams (53.3%) of sodium alginate are used instead of 82.5 grams.

### EXAMPLE 10

A dry mix of:

| Ingredients | grams | Percentage of dry powder |
|---|---|---|
| Sodium Alginate | 90 | 90% |
| Polyethylene glycol 3350 | 10 | 10% |

The ingredients are mixed in accordance with the method of Example 1 to produce a clear film coating.

### EXAMPLE 11

A dry mix of the ingredients below are mixed in accordance with the method of Example 1:

| Ingredients | grams | Percentage of dry powder |
|---|---|---|
| Sodium Alginate | 80 | 80% |
| Polyethylene glycol 3350 | 7 | 7% |
| FD&C Yellow No. 6 Aluminum Lake | 9 | 9% |
| Titanium dioxide | 4 | 4% |

A U.S.P. enteric test was used to assess the enteric efficiency of potassium iodide (300 mg.) tablets coated with the inventive enteric coating composition (approximately 1.3% w/w coating applied). The ingredients in potassium iodide (300 mg) tablet was blended in a V-Blender and compressed in a BB-2 tablet pressor. The compressed tablets were coated in an Accella Cota with inventive enteric coating solution. 150 tablets were examined in a disintegration tester for 1 hour, using 0.1 N HCl as the test medium at 37° C. to simulate gastric conditions. Subsequently, the tablet disintegration time in a buffer with a pH of 6.8 to simulate intestinal conditions was also evaluated.

Results indicated an ability to resist breakdown in gastric juice conditions for the prescribed period, while disintegration time in the buffer pH of 6.8 solution was 20-25 minutes (the time being 3-5 minutes for uncoated cores).

Another U.S.P. enteric test was used to assess the enteric efficiency of garlic (500 mg) tablets coated with the inventive enteric coating composition (approximately 1.3% w/w coating applied). 150 tablets were examined in a disintegration tester for 1 hour, using 0.1 N HCl as the test medium at 37° C. to simulate gastric conditions. Subsequently, the disintegration time in a buffer with a pH of 6.8 to simulate intestinal conditions was also evaluated.

Results indicated an ability to resist breakdown in gastric juice conditions for the prescribed period, while disintegration time in the buffer pH of 6.8 solution was 12-15 minutes (the time being 2-4 minutes for uncoated cores).

For a clear film coating, without pigments, the dry powder coating composition was made in an aqueous solution and applied to tablets cores. Tablets coated with the inventive coating solution have an intestinally soluble coating.

### EXAMPLE 12

The ingredients in the core portion, as noted below, are mixed together and pressed to form a tablet. This tablet is coated with an enteric coating composition according to Example 1, using a film coating process. The ingredients in the outer portion are mixed together. The enterically coated portion is placed within an aliquot of the outer portion and pressed to form a tablet wherein the enterically coated portion is surrounded by the outer portion. The tablet is optionally coated with a gelatin outer coating to enable easier swallowing.

| Component | Quantity (mg) | |
|---|---|---|
| Core portion | | |
| N-acetylcysteine | 525 | |
| Glutathione | 95 | |
| Vitamin E | 28 | IU |
| Vitamin C | 30 | |
| Microcrystalline | 208 | |
| Cellulose | | |
| Dicalcium Phosphate | 170 | |
| Dihydrate | | |
| Stearic Acid | 71.5 | |
| Croscarmellose Sodium | 33 | |
| Silicon Dioxide | 11 | |
| Magnesium Stearate | 11 | |
| Outer portion | | |
| N-acetylcysteine | 25 | |
| Glutathione | 5 | |
| Vitamin E | 2 | IU |
| Vitamin C | 2 | |
| Hydroxypropyl Cellulose | 1.8 | |
| Hydroxyl | 1.8 | |
| Methylcellulose | | |

### EXAMPLE 13

The components are combined, tabletted, and enterically coated according to Example 12. The formulation included the following components:

| Component | Quantity (mg) | |
|---|---|---|
| Core portion | | |
| N-acetylcysteine | 550 | |
| Glutathione | 100 | |
| Vitamin E | 30 | IU |
| Vitamin C | 30 | |
| Microcrystalline | 300 | |
| Cellulose | | |
| Dicalcium Phosphate | 110 | |
| Dihydrate | | |
| Stearic Acid | 69 | |
| Crospovidone | 35 | |
| Silicon Dioxide | 10 | |
| Magnesium Stearate | 11 | |
| Outer portion | | |
| N-acetylcysteine | 50 | |
| Glutathione | 5 | |
| Vitamin E | 2 | IU |
| Vitamin C | 2 | |
| Hydroxyl | 3.0 | |
| Methylcellulose | | |
| Hydroxypropyl | 0.6 | |
| Cellulose | | |

### EXAMPLE 14

The components below are combined together and mixed to form a generally uniform mixture. The mixture is tabletted, and the tablets coated using an enteric coating composition according to Example 11. using a film coating technique.

| Component | Quantity (mg) | |
|---|---|---|
| N-acetylcysteine | 550 | |
| Glutathione | 100 | |
| Vitamin E | 30 | IU |
| Vitamin C | 30 | |
| Microcrystalline Cellulose | 208 | |
| Dicalcium Phosphate | 170 | |
| Dihydrate | | |
| Stearic Acid | 71.5 | |
| Croscarmellose Sodium | 33 | |
| Silicon Dioxide | 11 | |
| Magnesium Stearate | 11 | |

### EXAMPLE 15

The components are combined and tabletted according to Example 14, except that no enteric coating was used. The formulation included the following components:

| Component | Quantity (mg) | |
|---|---|---|
| N-acetylcysteine | 550 | |
| Glutathione | 100 | |
| Vitamin E | 30 | IU |
| Vitamin C | 30 | |
| Microcrystalline Cellulose | 208 | |
| Dicalcium Phosphate | 170 | |
| Dihydrate | | |
| Stearic Acid | 71.5 | |
| Croscarmellose Sodium | 33 | |
| Silicon Dioxide | 11 | |
| Magnesium Stearate | 11 | |

### EXAMPLE 16

The components are combined, tabletted, and enterically coated according to Example 12. The formulation includes the following components:

| Component | Quantity (mg) | |
|---|---|---|
| Core portion | | |
| N-acetylcysteine | 6000 | |
| Glutathione | 600 | |
| Vitamin E | 120 | IU |
| Vitamin C | 100 | |
| Microcrystalline | 4016 | |
| Cellulose | | |
| Dicalcium Phosphate | 3400 | |
| Dihydrate | | |
| Stearic Acid | 1430 | |
| Croscarmellose Sodium | 660 | |
| Silicon Dioxide | 220 | |
| Magnesium Stearate | 220 | |
| Outer portion | | |
| N-acetylcysteine | 500 | |
| Glutathione | 71 | |
| Vitamin E | 40 | IU |
| Vitamin C | 40 | |
| Hydroxypropyl Cellulose | 36 | |
| Hydroxyl | 36 | |
| Methylcellulose | | |

### EXAMPLE 17

The components are combined, tabletted, and enterically coated according to Example 12. The formulation includes the following components:

| Component | Quantity (mg) | |
|---|---|---|
| Core portion | | |
| N-acetylcysteine | 2500 | |
| Glutathione | 4500 | |
| Vitamin E | 560 | IU |
| Vitamin C | 600 | |
| Microcrystalline | 4016 | |
| Cellulose | | |
| Dicalcium Phosphate | 3400 | |
| Dihydrate | | |
| Stearic Acid | 1430 | |
| Croscarmellose Sodium | 660 | |
| Silicon Dioxide | 220 | |
| Magnesium Stearate | 220 | |
| Outer portion | | |
| N-acetylcysteine | 500 | |
| Glutathione | 100 | |
| Vitamin E | 40 | IU |
| Vitamin C | 40 | |
| Hydroxypropyl Cellulose | 36 | |
| Hydroxyl | 36 | |
| Methylcellulose | | |

Many modifications and variations of the embodiments described herein may be made without departing from the scope, as is apparent to those skilled in the art. The specific embodiments described herein are offered by way of example only.

The various compounds and methods described above provide a number of ways to carry out the invention. Of course, it is to be understood that not necessarily all objectives or advantages described may be achieved in accordance with any particular embodiment described herein. Thus, for example, those skilled in the art will recognize that the compounds may be made in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and components discussed above, as well as other known equivalents for each such feature or component, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A pharmaceutical preparation, comprising:
a solid form comprising an active ingredient; and
one or more coating layers, wherein at least one coating layer is an enteric layer formed from a solution consisting essentially of water, sodium alginate and a water-soluble plasticizer.

38. A pharmaceutical preparation according to claim 37, wherein the enteric layer is at least partially disposed on the solid form comprising an active ingredient.

39. A pharmaceutical preparation according to claim 37, wherein at least one coating layer is disposed between the enteric layer and the solid form comprising an active ingredient.

40. A pharmaceutical preparation according to claim 37, wherein at least one coating layer comprises a pharmaceutically acceptable carrier.

41. A pharmaceutical preparation according to claim 37, wherein at least one coating layer comprises an active ingredient.

42. A pharmaceutical preparation according to claim 37, wherein the water-soluble plasticizer comprises polyalkylene glycol, polyalkylene glycol copolymer, polyalkylene oxide, and/or polyalkylene oxide copolymer.

43. A pharmaceutical preparation according to claim 37, wherein the water-soluble plasticizer comprises polyethylene glycol.

44. A pharmaceutical preparation according to claim 37, wherein the solution includes one or more substances chosen from the group consisting of pigment particles, coloring agents and anti-caking agents.

45. A pharmaceutical preparation according to claim 37, wherein the sodium alginate comprises about 50-95% by weight of all the particles in the solution.

46. A pharmaceutical preparation according to claim 37, wherein the sodium alginate comprises about 75-85% by weight of all the particles in the solution.

47. A pharmaceutical preparation according to claim 44, where in the pigment particles, coloring agents and anti-caking agents comprise about 0.1-15% by weight of all the particles in the solution.

48. A pharmaceutical preparation according to claim 44, wherein the sodium alginate comprises about 50-95% by weight of all the particles in the solution.

49. A pharmaceutical preparation according to claim 44, wherein the sodium alginate comprises about 75-85% by weight of all the particles in the solution.

50. A pharmaceutical preparation according to claim 46, wherein the water-soluble plasticizer comprises about 1-50% by weight of all the particles in the solution.

51. A pharmaceutical preparation according to claim 46, wherein the water-soluble plasticizer comprises about 5-25% by weight of all the particles in the solution.

52. A pharmaceutical preparation according to claim 48, wherein the water-soluble plasticizer comprises about 1-50% by weight of all the particles in the solution.

53. A pharmaceutical preparation according to claim 48, wherein the water-soluble plasticizer comprises about 5-25% by weight of all the particles in the solution.

54. A tablet comprising:
a first portion comprising an active ingredient; and
an enteric layer, wherein the enteric layer consists essentially of sodium alginate and a plasticizer.

55. A tablet according to claim 54, wherein the enteric layer is at least partially disposed on the first portion comprising an active ingredient.

56. A tablet according to claim 54, further comprising a coating layer disposed at least partially between the enteric layer and the first portion comprising an active ingredient.

57. A tablet according to claim 54, further comprising a coating layer comprising a pharmaceutically acceptable carrier.

58. A tablet according to claim 54, wherein the plasticizer comprises polyalkylene glycol, polyalkylene glycol copolymer, polyalkylene oxide, and/or polyalkylene oxide copolymer.

59. A tablet according to claim 54, wherein the plasticizer comprises polyethylene glycol.

60. A tablet according to claim 54, wherein the enteric layer includes one or more substances selected from the group consisting of pigment particles, coloring agents and anti-caking agents.

61. A tablet according to claim 54, wherein the enteric layer is formed from a solution consisting essentially of water, sodium alginate and a water-soluble plasticizer.

62. A tablet according to claim 54, where in the sodium alginate comprises about 50-95% by weight of the enteric layer.

63. A tablet according to claim 54, wherein the sodium alginate comprises about 75-85% by weight of the enteric layer.

64. A tablet according to claim 62, wherein the plasticizer comprises about 1-50% by weight of the enteric layer.

65. A tablet according to claim 63, wherein the plasticizer comprises about 1-50% by weight of all the particles in the solution.

66. A layer disposed on at least a portion of a solid form pharmaceutical preparation for oral administration to a mammal, wherein the layer consists essentially of sodium alginate and a plasticizer.

67. A layer according to claim 66, wherein the plasticizer comprises polyalkylene glycol, polyalkylene glycol copolymer, polyalkylene oxide, and/or polyalkylene oxide copolymer.

68. A layer according to claim 66, wherein the plasticizer comprises polyethylene glycol.

69. A layer according to claim 66, further comprising one or more substances selected from the group consisting of pigment particles, coloring agents and anti-caking agents.

70. A layer according to claim 66, wherein the layer is formed from a solution consisting essentially of water, sodium alginate and a water-soluble plasticizer.

71. A layer according to claim 66, wherein the sodium alginate comprises about 50-95% by weight of the layer.

72. A layer according to claim 66, wherein the water-soluble plasticizer comprises about 1-50% by weight of the layer.
